(19) United States
(12) Patent Application Publication
(10) Pub. No.: US 2016/0033788 A1
(43) Pub. Date: Feb. 4, 2016

(54) EYEGLASSES FOR BLOCKING ALLERGENS

(71) Applicant: Manish Chadha, Kew Gardens, NY (US)

(72) Inventor: Manish Chadha, Kew Gardens, NY (US)

(21) Appl. No.: 14/813,027

(22) Filed: Jul. 29, 2015

Related U.S. Application Data
(60) Provisional application No. 62/030,267, filed on Jul. 29, 2014.

(51) Int. Cl. G02C 5/00; G02C 7/12; G02C 5/12; A61F 9/02

(52) U.S. Cl. CPC G02C 5/001; A61F 9/026; G02C 7/12; G02C 5/122

(57) ABSTRACT

Exemplary embodiments of eyeglasses are provided that axe configured to block allergens from a user's eyes, having a frame having lenses that are polarized, an allergen blocking top cover extending from the frame in a direction towards a user's forehead configured to block allergens from entering a top portion of a user's eyes, a forehead seal extending from the allergen blocking top cover to a user's forehead configured to block allergens from entering the top portion of a user's eyes, a first allergen blocking peripheral cover extending from a first side portion of the frame extending towards a user's face configured to block allergens from entering a first side portion of a user's eyes, and a second allergen blocking peripheral cover extending from a second side portion of the frame extending towards a user's face configured to block allergens from entering a second side portion of a user's eyes.

### CROSS-REFERENCE TO RELATED APPLICATIONS

This application relates to and claims priority from U.S. Provisional Patent Application Ser. No. 62/030,267 filed Jul. 29, 2014, the entire disclosure of which is hereby incorporated herein by reference.

### FIELD OF THE DISCLOSURE

The present disclosure relates to exemplary embodiments of eyeglasses, and more particularly, to exemplary embodiments of eyeglasses for blocking allergens.

### BACKGROUND INFORMATION

Children and adults often get allergies from various allergens entering their eyes. Medicine is available for children and adults, but only alter the allergen has entered their eyes. These medications also often have side effects, which can be harmful. It would be useful to provide an apparatus that could prevent allergies by preventing the allergens from entering a user's eyes.

At least some of the above described problems can be addressed by exemplary embodiments of the methods and systems according to the present disclosure. Exemplary embodiments of an apparatus is provided for blocking allergens.

In some exemplary embodiments, an apparatus for blocking allergens from a user's eyes can be provided, comprising a frame having lenses that are polarized, an allergen blocking top cover extending from the frame in a direction towards a user's forehead configured to block allergens from entering a top portion of a user's eyes; a forehead seal extending from the allergen blocking top cover to a user's forehead configured to block allergens from entering the lop portion of a user's eyes, a first allergen blocking peripheral cover extending from a first side portion of the frame extending towards a user's face configured to block allergens from entering a first side portion of a user's eyes, and a second allergen blocking peripheral cover extending from a second side portion of the frame extending towards a user's face configured to block allergens from entering a second side portion of a user's eyes. The apparatus can further comprise an adjustable and flexible nose pad mounted to the frame.

In some exemplary embodiments, the apparatus can further comprise a first elongated side extending from the frame adjacent to the allergen blocking top cover on a top side and adjacent to the first allergen blocking peripheral cover on a bottom side, and a second elongated side extending from the frame adjacent to the allergen blocking top cover on a top side and adjacent to the second allergen blocking peripheral cover on a bottom side. The apparatus can further comprise a first arm extending from and pivotally connected to the first elongated side, and a second arm extending from and pivotally connected to the second elongated side. The apparatus can further comprise a first ear piece extending from the first arm and configured for placement on a user's ear, and a second ear piece extending from the second arm and configured for placement on a user's ear.

In some exemplary embodiments, the allergen blocking top cover and the first and second allergen blocking peripheral covers can be comprised of TR90. The forehead seal, nose pad, first ear piece and second ear piece can be comprised of rubber. The frame and the first and second elongated sides can be comprised of polycarbonate.

### BRIEF DESCRIPTION OF THE DRAWINGS

The foregoing and other objects of the present disclosure will be apparent upon consideration of the following detailed description, taken in conjunction with the accompanying exemplary drawings and claims, in which like reference characters refer to like parts throughout, and in which:

FIG. 1(a) is a perspective view of eyeglasses in an open configuration according to a first exemplary embodiment of the present disclosure;

FIG. 1(b) is a top view of eyeglasses in an open configuration according to a first exemplary embodiment of the present disclosure;

FIG. 1(c) is a front view of eyeglasses in an open configuration according to a first exemplary embodiment of the present disclosure;

FIG. 1(d) is a side view of eyeglasses in an open configuration according to a first exemplary embodiment of the present disclosure;

FIG. 2(a) is a perspective view of eyeglasses in a closed configuration according to a first exemplary embodiment of the present disclosure;

FIG. 2(b) is a top view of eyeglasses in a closed configuration according to a first exemplary embodiment of the present disclosure;

FIG. 2(c) is a front view of eyeglasses in a closed configuration according to a first exemplary embodiment of the present disclosure;

FIG. 2(d) is a side view of eyeglasses in a closed configuration according to a first exemplary embodiment of the present disclosure;

FIG. 3 is a rear perspective view of eyeglasses in an open configuration according to a first exemplary embodiment of the present disclosure;

FIG. 4 is an illustration of a user wearing eyeglasses according to a first exemplary embodiment of the present disclosure;

FIG. 5(a) is a perspective view of eyeglasses in an open configuration according to a second exemplary embodiment of the present disclosure;

FIG. 5(b) is a top view of eyeglasses in an open configuration according to a second exemplary embodiment of the present disclosure;

FIG. 5(c) is a front view of eyeglasses, in an open configuration according to a second exemplary embodiment of the present disclosure;

FIG. 5(d) is a side view of eyeglasses in an open configuration according to a second exemplary embodiment of the present disclosure;

FIG. 6 is an illustration of a user wearing eyeglasses according to a second exemplary embodiment of the present disclosure;

FIG. 7(a) is a perspective view of eyeglasses in an open configuration according to a third exemplary embodiment of the present disclosure;

FIG. 7(b) is a top view of eyeglasses in an open configuration according to a third exemplary embodiment of the present-disclosure;

FIG. 7(c) is a front view of eyeglasses in an open configuration according to a third exemplary embodiment of the present disclosure;

FIG. 7(d) is a side view of eyeglasses in an open configuration according to a third exemplary embodiment of the present disclosure; and

FIG. 8 is an illustration of a user wearing eyeglasses according to a third exemplary embodiment of the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described embodiments without departing from the true scope and spirit of the subject disclosure.

### DETAILED DESCRIPTION OF EXEMPLARY EMBODIMENT DISCLOSURE

Exemplary embodiments of the eyeglasses of the present disclosure will now be described with reference to the figures. The following description of the various embodiments is merely exemplary in nature and is in no way intended to limit the scope of the disclosure, its application, or uses.

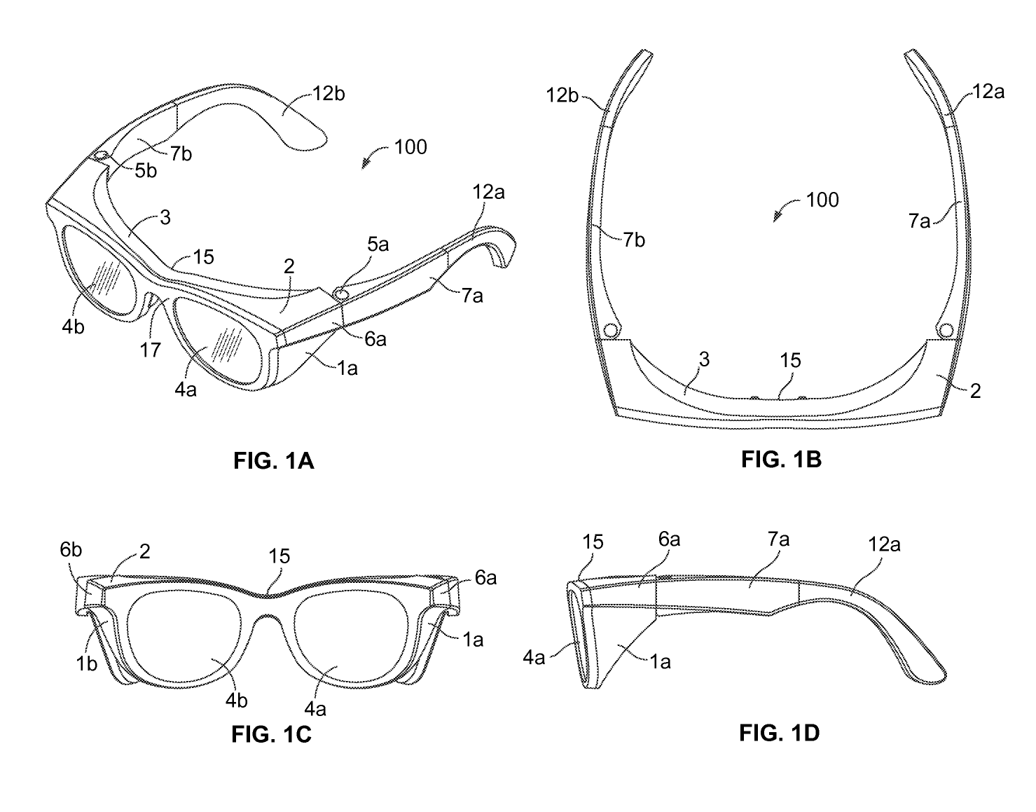

FIG. 1(a) is a perspective view of eyeglasses in an open configuration according to a first exemplary embodiment of the present disclosure. FIG. 1(b) is a top view, FIG. 1(c) is a front view and FIG. 1(d) is a side view thereof. The eyeglasses 100 can have a first arm 7a and a second arm 7b for placement of the eyeglasses 100 on a user. The first arm 7a and the second arm 7b can have an ear piece 12a and an ear piece 12b, respectively, at a first end. The arms and earpieces can vary in shape and size to accommodate different head sizes. The arms 7a, 7b can be made separate or integral with the ear pieces 12a, 12b. In some exemplary embodiments, the first and second arms 7a, 7b cars be made of a first material, such as TR90, and the ear pieces can be made of a second material, such as a deformable or hardened wire (which can include a top rubber coating).

The first arm 7a and second arm 7b can be connected to a front portion 15 at a second end opposite the first end connected to the ear pieces 12a and 12b. Various known mechanical fasteners can be used for connecting the first arm 7a and the second arm 7b to the front portion 15. In some exemplary embodiments, a hinge 5a and 5b can be provided to connect the first arm 7a and second arm 7b, respectively, to the front portion 15.

The front portion 15 of the eyeglasses 100 can have an allergen blocking top cover 2, and allergen blocking peripheral covers 1a and 1b. An elongated side 6a can be provided between the allergen blocking top cover 2 and allergen blocking peripheral cover 1a, and an elongated side 6b can be provided between the allergen blocking top cover 2 and allergen blocking peripheral cover 1b. A forehead seal 3 can be provided on the from portion, which can adjoin the allergen blocking top cover 2. The forehead seal 3 can be made from a soft, hypo-allergenic material so as to not irritate the user's skin, while providing a comfortable seal against the user's forehead. Optic lenses 4a and 4b can be provided, within a lens frame 17 in the front portion 15. The lenses 4a and 4b can provide protection from the sun (e.g., colored, darkened or polarized lenses) and can be built into the lens frame 17 at the time of manufacturing.

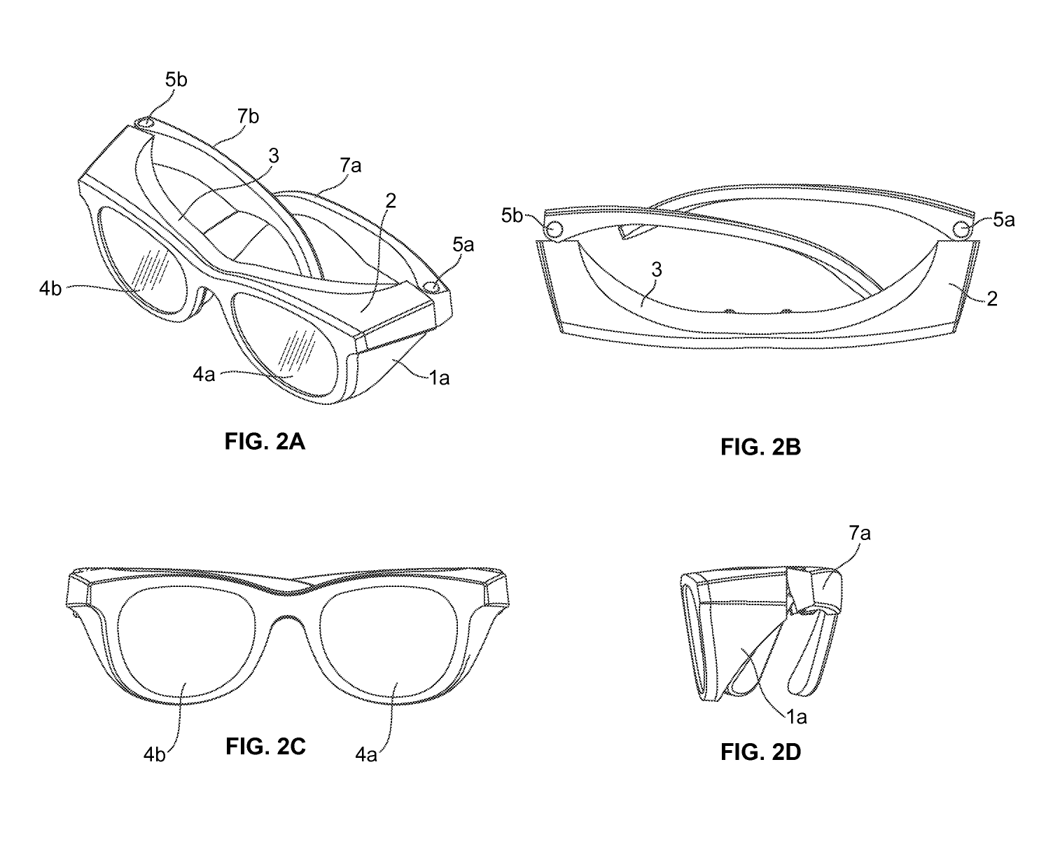

FIG. 2(a) is a perspective view of eyeglasses in a closed configuration according to a first exemplary embodiment of the present-disclosure. FIG. 2(b) is a top view, FIG. 2(c) is a front view and FIG. 2(d) is a side view thereof. As can be seen in Figures 2(a)-2(d), the hinges 5a and 5b allow the first arm 7a and second arm 7b, respectively, to rotate to a closed configuration when the eyeglasses 100 are to be stored, packaged or put away.

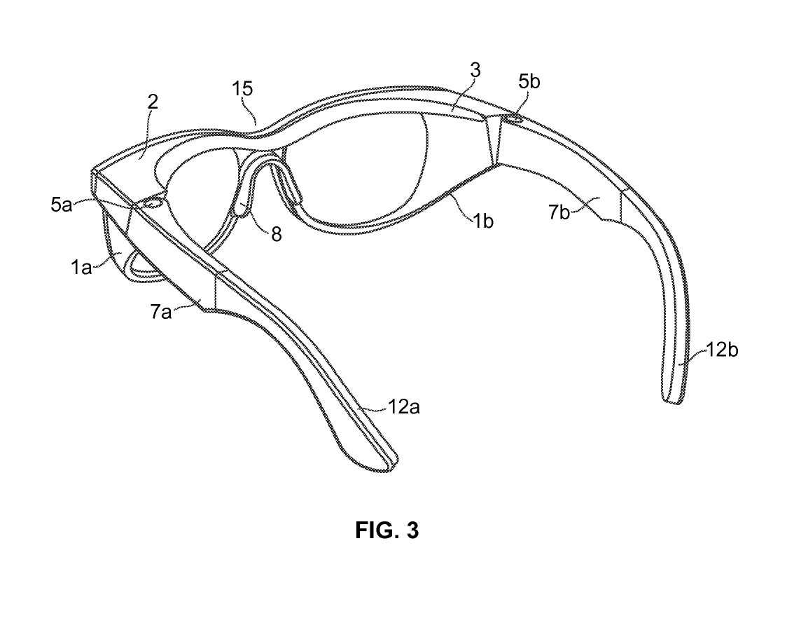

FIG. 3 is a rear perspective view of eyeglasses in an open configuration according to a first exemplary embodiment of the present disclosure. As shown in FIG. 3, the forehead seal 3 can be provided along the allergen blocking top cover 2 on a top portion of the front portion 15. An enlarged nose pad 8 can be provided on the front portion 15 to rest on a user's nose and provide an additional seal against allergens. The nose pad 8 can be made of a flexible material and can be adjusted.

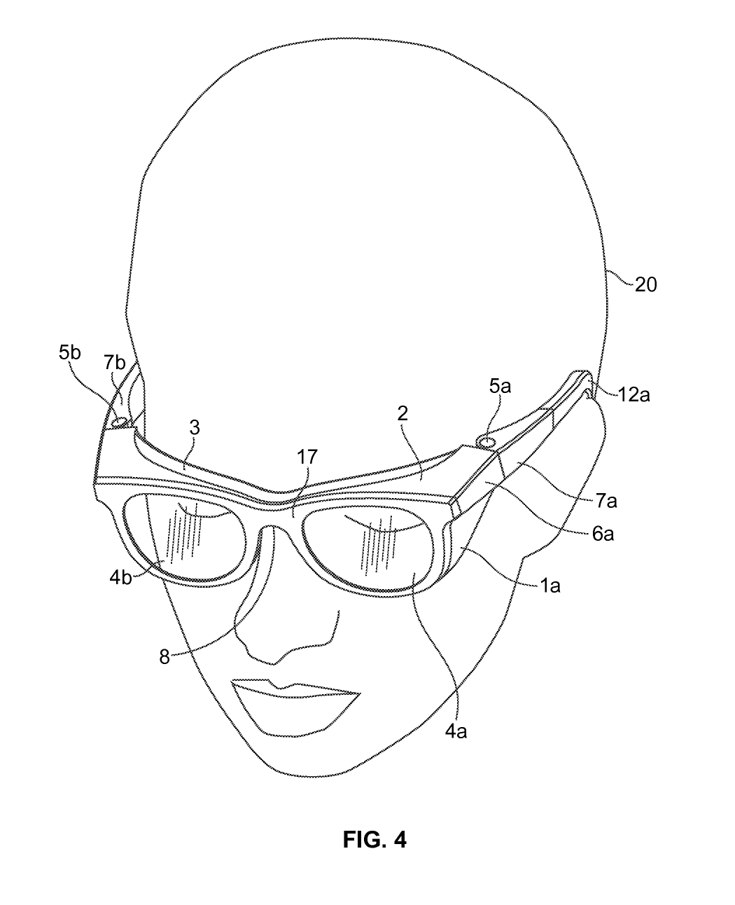

FIG. 4 is an illustration of a user 20 wearing the eyeglasses 100 according to a first exemplary embodiment of the present disclosure. As shown in FIG. 4, the allergen blocking top cover 2 provides protection along a top portion of a user's eyes between the eyes and the lens frame 17 holding the lenses 4a and 4b. A forehead seal 3 can touch or press against the forehead and provide a further seal against allergens between the allergen blocking top cover 2 and the user's forehead. Allergen blocking peripheral covers 1a and 1b can touch or press against a user's face, or substantially cover the area between the frame 17 and the user's face, and provide peripheral seals between the user's face and the lens frame 17 to block allergens from entering the eyes of the user 20. The nose pad seal 8 can touch or press against the user's forehead and/or nose, and provide a seal between the lens frame 17 and the user's face, blocking allergens from entering the user's eyes. The elongated sides 6a and 6b provided also provide protection against allergens between the lens frame 17 and the user's eyes.

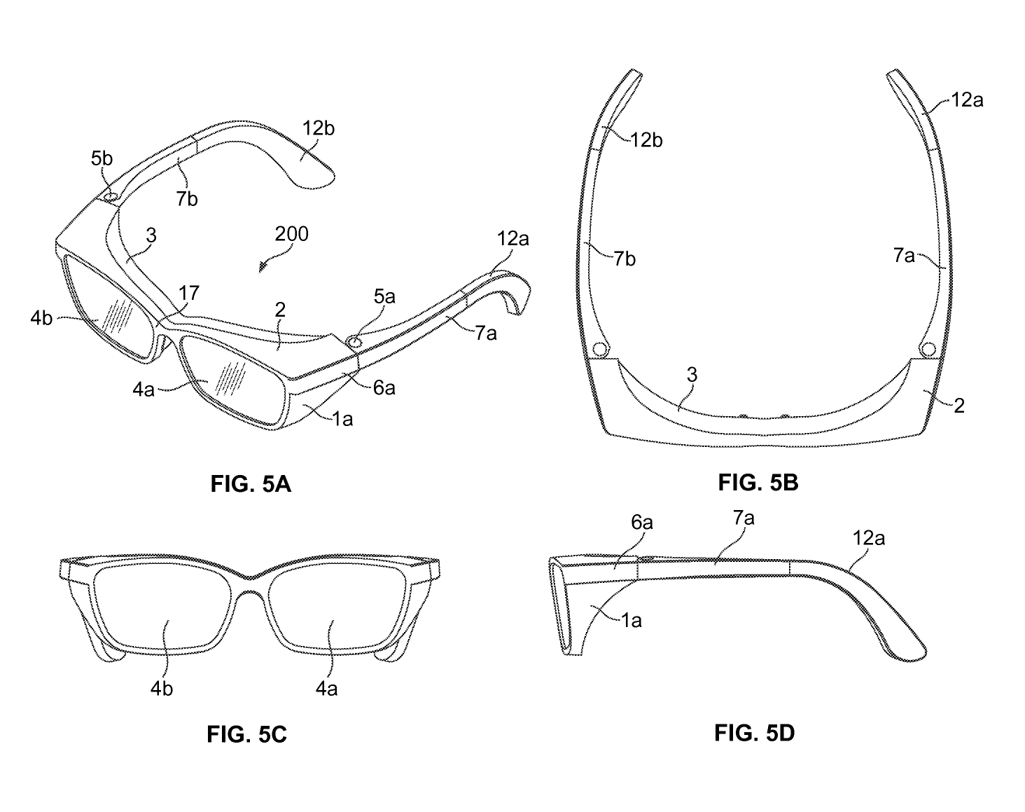

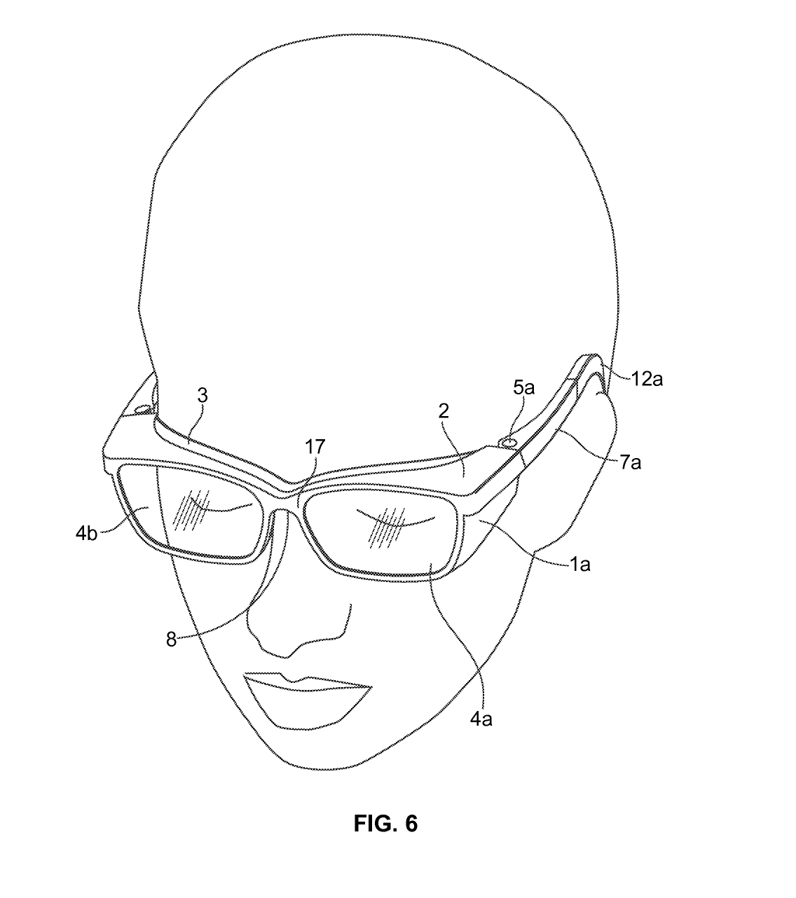

Various other embodiments of the parts of the eyeglasses 100 are contemplated by the present disclosure. For example, the frames 17 can have various shapes, sizes and configurations. The lenses 4a and 4b can have various shapes and sizes, and can be prescription lenses or sunglasses. The eyeglasses 100 can be provided as sunglasses of various styles, such as but not limited to rounded, circular or oval shapes. Various styles and sizes can be provided for the first and second arms, as well as the ear pieces. For example, FIGS. 5(a)-5(d) illustrate a second exemplary embodiment of the present disclosure, providing eyeglasses 200 with prescription lenses 4a and 4b, as well as different shapes of the earpieces 12a and 12b, first and second arms 7a and 7b, allergen blocking top cover 2, allergen blocking peripheral covers 1a and 1b, and elongated sides 6a and 6b. As shown in FIG. 6, a user can wear the eyeglasses with a different configuration and still have protection from allergens as described above with respect to FIGS. 1-4.

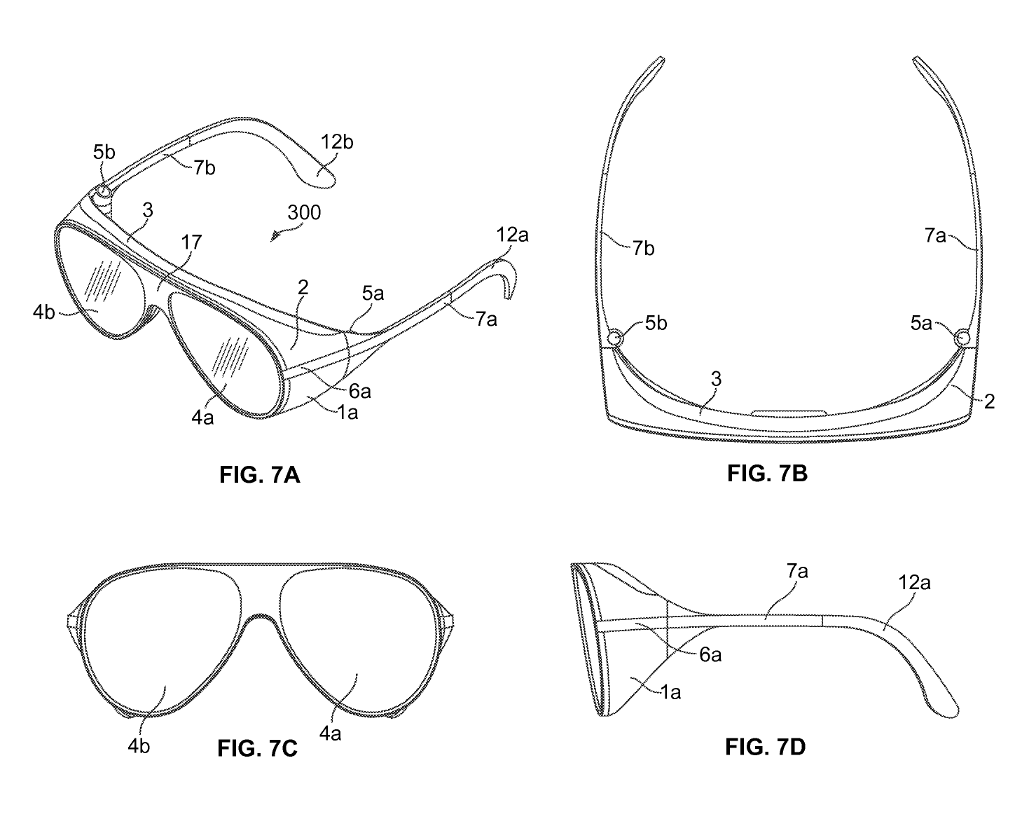

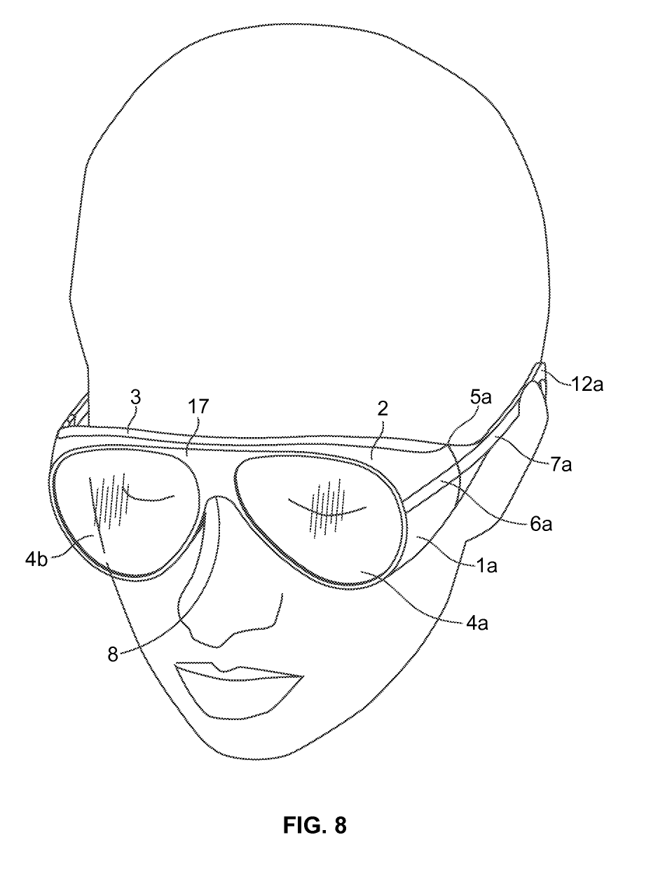

As shown in FIGS. 7(a)-7(d), which illustrate a third exemplary embodiment of the present disclosure, eyeglasses 300 can be provided having different shapes of the lenses 4a and 4b, such as a round style. As shown in FIG. 8, a user can wear the eyeglasses 300 with a different shape of the lenses and still have protection from allergens as described above with respect to FIGS. 1-4.

Various materials can be used for the various parts of the eyeglasses 100. For example, the allergen blocking top cover 2 and the allergen blocking peripheral covers 1a and 1b can be made of a thermoplastic material, such as TR90. TR90 can provide various advantages, such as but not limited to being durable, resilient to damage, flexible, pliable and lightweight. Eyeglasses made with TR90 can be flexible and can bend under pressure and contour a user's face comfortably. They are lighter than other materials typically used for eyeglasses, such as acetate cellulose plastic. They are also suitable for various climates, and can be resistant to high temperatures and not easily melt or burn. The forehead seal 3, earpieces 12a and 12b, and nose pad 8 can be made of a soft material, such as rubber or plastic. The frame 17 and the elongated sides 6a, 6b can be made of polycarbonate, metal steel aluminum, titanium, flexon or nylon, or a composite of two or more of these materials. The exemplary embodiments of the eyeglasses described herein are not limited to any particular type of material.

Various other consideration can also be addressed in the exemplary embodiments of the present disclosure. The exemplary embodiments of the present disclosure can be provided in various eyeglasses, such as sunglasses or prescription glasses. Various measurements can be provided, such as varying lengths, widths and thicknesses of all the parts, including the frame, lenses and arms. Difference styles, shapes and configurations of the eyeglasses can be provided. Various materials can be used for the frames, arms and ear pieces, such as plastic, metal or composite materials. Different type of material can be used for the lenses, e.g., glass, plastic or any composite material(s). The exemplary embodiments of the present disclosure are not limited to any type-of material, size or shape.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, manufacture and methods which, although not explicitly shown or described herein, embody the principles of the disclosure and are thus within the spirit and scope of the disclosure. The disclosures of all systems, documents and publications cited herein are hereby incorporated herein by reference in their entireties.

What is claimed is:

1. An apparatus for blocking allergens from a user's eyes, comprising:
a frame having lenses that are polarized;
an allergen blocking top cover extending from, the frame in a direction towards a user's forehead configured to block allergens from entering a top portion of a user's eyes;
a forehead seal extending from the allergen blocking top cover to a user's forehead configured to block allergens from entering the top portion of a user's eyes;
a first allergen blocking peripheral cover extending from a first side portion of the frame extending towards a user's face configured to block allergens from entering a first side portion of a user's eyes; and
a second allergen blocking peripheral cover extending from a second side portion of the frame extending towards a user's face configured to block allergens from entering a second side portion of a user's eyes.

2. The apparatus of claim 1, further comprising:
an adjustable and flexible nose pad mounted to the frame.

3. The apparatus of claim 2, further comprising:
a first elongated, side extending from the frame adjacent to the allergen blocking top cover on a top side and adjacent to the first allergen blocking peripheral cover on a bottom side; and
a second elongated side extending from the frame adjacent to the allergen blocking top cover on a top side and adjacent to the second allergen blocking peripheral cover on a bottom side.

4. The apparatus of claim 3, further comprising:
a first arm extending from and pivotally connected to the first elongated side; and
a second arm extending from and pivotally connected to the second elongated side.

5. The apparatus of claim 4, further comprising:
a first ear piece extending from the first arm and configured for placement on a user's ear; and
a second ear piece extending from the second arm and configured for placement on a users ear.

6. The apparatus of claim 5, wherein the allergen blocking top cover and the first and second allergen blocking peripheral covers are comprised of TR90.

7. The apparatus of claim 6, wherein the forehead seal, nose pad, first ear piece and second ear piece are comprised of rubber.

8. The apparatus of claim 7, wherein the frame and the first and second elongated sides are comprised of polycarbonate.
